(19) United States
(12) Patent Application Publication
(10) Pub. No.: US 2025/0391104 A1
(43) Pub. Date: Dec. 25, 2025

(54) SYNTHESIS OF IMAGES FOR 2D TO 3D ASYMMETRIC FEATURE PRESERVATION

(71) Applicant: KONINKLIJKE PHILIPS N.V., Eindhoven (NL)

(72) Inventors: PRAVEEN KUMAR PANDIAN SHANMUGANATHAN, MONROEVILLE, PA (US); NITINRAJ NAIR, PITTSBURGH, PA (US)

(21) Appl. No.: 17/988,349

(22) PCT Filed: Nov. 8, 2022
(86) PCT No.: PCT/EP2022/081150

§ 371 Date: Nov. 16, 2022

(51) Int. Cl. G06T 17/00; G06T 7/593

(52) U.S. Cl. CPC G06T 17/00; G06T 7/593; G06T 2207/10012; G06T 2207/20081; G06T 2207/20084; G06T 2207/30201

(57) ABSTRACT

An embodiment provides a method of producing a three-dimensional (3D) model of an object based on a single, frontal input two-dimensional (2D) image. In one example a method includes obtaining an actual, frontal 2D image of an object and generating a pair of synthetic 2D multiview images of the object based on the actual, frontal 2D image of the object. A 3D model of the object is produced based on at least the pair of synthetic 2D multiview images. The 3D model conserves an asymmetry of the object. An output using the 3D model of the object is produced that conserves the asymmetry.

### CROSS-REFERENCE TO RELATED APPLICATIONS

This patent application claims the priority benefit under 35 U.S.C. § 119(e) of U.S. Provisional Application No. 63/280,667, filed on Nov. 18, 2021, the contents of which are herein incorporated by reference.

### BACKGROUND OF THE INVENTION

### 1. Field of the Invention

The present invention pertains to techniques for generating a three-dimensional (3D) model of an object using a two-dimensional (2D) image input. Some of the subject matter relates to using an asymmetry preserving 3D model in biometrics applications.

### 2. Description of the Related Art

Conventional approaches to generating a 3D model of an object, e.g., a user's face, include use of 3D scanning techniques or use of multiple 2D input images offering different angles of the object (multi-view images). While 3D scanning tools offer accurate data for generating a 3D model of the object, obtaining the 3D scan data requires use of complex and often expensive equipment. Similarly, while capturing 2D multi-view images of the object with a camera may be used to generate an accurate 3D model, many typical use scenarios make this approach unworkable in practice, for example, because many end users have difficulty capturing the required input images to generate a sufficiently accurate 3D model.

Some progress has been made in synthesizing 2D multi-view images from an original 2D input image, such as for example as described in KR 102245220 B1, entitled “Apparatus for reconstructing 3d model from 2d images based on deep-learning and method thereof,” published Apr. 27, 2021. However, as further described herein, a need remains for improved techniques for generating a 3D model using a 2D image input, particularly in relation to certain application spaces where 3D model accuracy is important.

### SUMMARY OF THE INVENTION

Conventionally, complex and expensive 3D scanning is used to obtain 3D modelling data for an object, e.g., a user's face. However, this approach is not useful in many contexts and even though some advances have been made in using 2D images for creating multiview images for modelling 3D objects, a need exists for facilitating use of readily accessible hardware devices to implement accurate 3D modelling based on a simple to capture 2D image.

Accordingly, it is an advantage of the claimed embodiments to provide model(s) that operate on 2D imagery to produce an accurate 3D model of an object, including preservation of any asymmetric features of the object. Embodiments facilitate this process by utilizing an automated technique for creation or synthesis of specific 2D multiview images using a single 2D image input. The specific 2D multiview images are synthesized to include asymmetry preserving views of the object, for example having angular offsets from the 2D frontal input image that offer a wide-angle view of the object type, such as a face, that capture relevant asymmetries of the object. In an embodiment, the 2D mutltiview images that are synthesized comprise a stereo pair formed from a single 2D frontal input image, e.g., captured using conventional hardware such as a smartphone camera or other readily available image sensor.

In summary, one embodiment provides a method including obtaining, using a set of processors, an actual, frontal 2D image of an object. The method generates a pair of synthetic 2D multiview images of the object based on the actual, frontal 2D image of the object and produces a 3D model of the object based on at least the pair of synthetic 2D multiview images. The 3D model conserves an asymmetry of the object. The method includes providing an output using the 3D model of the object that conserves the asymmetry.

Another embodiment provides a device, such as a user's smartphone, tablet computing device, or other client device that is provided with a trained artificial neural network that performs the methods of using 2D multiview images as described herein.

A further embodiment provides a cloud or server-based device or system that acts to train and/or implement an artificial neural network that performs the methods of using 2D multiview images as described herein.

A yet further embodiment provides a computer readable program product for implementing methods related to forming and using 2D multiview images as described herein.

The foregoing is a summary and thus may contain simplifications, generalizations, and omissions of detail; consequently, those skilled in the art will appreciate that the summary is illustrative only and is not intended to be in any way limiting.

These and other objects, features, and characteristics of the present invention, as well as the methods of operation and functions of the related elements of structure and the combination thereof, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is an example pipeline architecture for synthesis of images for asymmetric feature preservation;

FIG. 2 is an example of a method of using multi-view images for loss or error correction in training;

FIG. 3 is an example of a method of using global loss or error correction in training;

FIG. 4 is an example of a method of using a frontal 2D image to form 2D multiview images for 3D modeling;

FIG. 4A provides an example of a method of using a 3D model output;

FIG. 4B provides another example of a method of using a 3D model output; and

FIG. 5 is a diagram of example system components.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “operatively coupled” means that two or more elements are coupled so as to operate together or are in communication, unidirectional or bidirectional, with one another. As used herein, the term “number” shall mean one or an integer greater than one (i.e., a plurality). As used herein a “set” shall mean one or more.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well known structures, materials, or operations are not shown or described in detail to avoid obfuscation.

Obtaining data representative of an object's three-dimensional (3D) geometry is important to extract to allow for accurate measurements for any design or sizing application in different industries. However, conventionally to extract accurate 3D measurements, 3D scanners have to be in place which are more expensive and difficult to operate. An embodiment provides a solution to extract 3D meshes using a two-dimensional (2D) image. This permits essentially any 2D image, e.g., a red, green, bule (RGB) 2D image from a smartphone camera, to be provided to an embodiment to achieve a 3D mesh with near-equal quality as compared with meshes formed using 3D scanners.

It will be more fully appreciated by reference to this detailed description, its examples, and the associated drawings that multi-view 2D images are accurate and effective ways to reconstruct a 3D model of an object. However, conventionally obtaining or extracting a useful multi-view 2D images needs special hardware and protocols to make use of these to form a meaningful 3D reconstruction. While certain advances have been made in synthetically creating 2D images from a single, actual input image for 3D object modelling, these approaches do not generate 3D models having the required accuracy for many applications, in part because prior techniques do not take care to provide for generation of requisite 2D multiview images, for example wide-angle or stereo pairs of synthetic 2D images for face modelling.

The example embodiments describe methods of extracting or synthesizing a pair of 2D images suitable for use in accurate 3D object modeling that conserves asymmetry in the object, such as useful in facial modeling for biometric security as well as accurate form and fit applications used in connection with medical devices. For example, an embodiment generates a predetermined pair of 2D multiview images, such as a stereo pair of synthetic 2D images having a predetermined angular offset, from one single actual 2D image input, without any special hardware. One example method uses an artificial neural network that takes in an actual, frontal 2D image and recreates a left and right stereo pair of synthetic 2D images for 3D model reconstruction. The resultant 3D model may be used for various purposes, including but not limited to evaluating the original input 2D image for certain features, such as requisite biometric features for a security application, relevant biometric features that adjust a fit of a biomedical device, etc.

The description now turns to the figures. The illustrated example embodiments will be best understood by reference to the figures. The following description is intended only by way of example, and simply illustrates certain example embodiments.

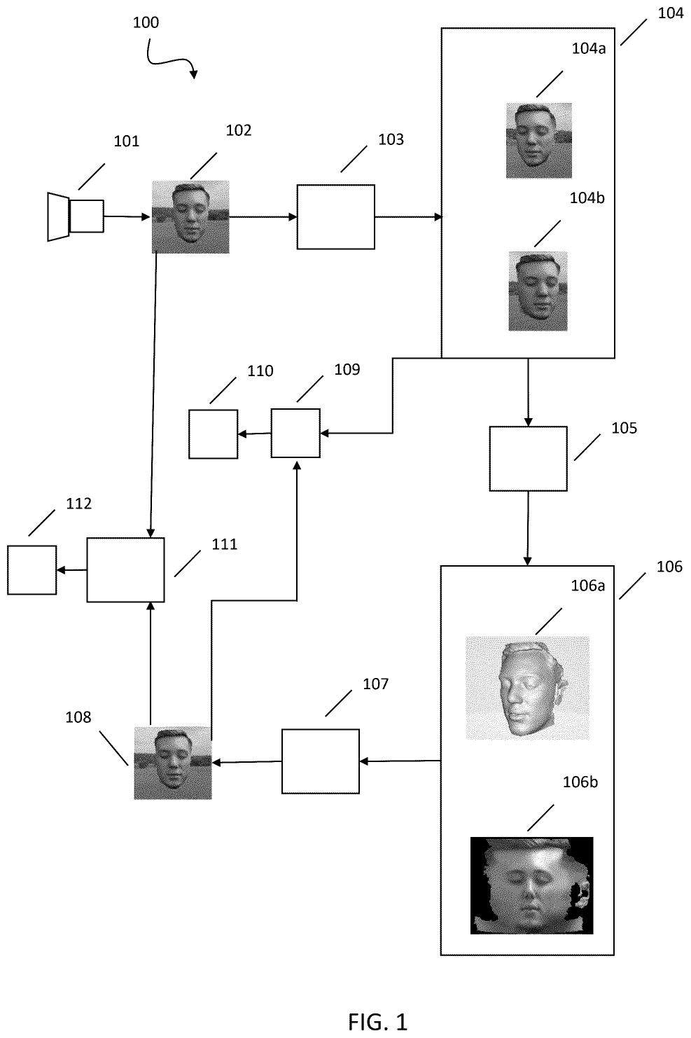

FIG. 1 schematically illustrates an example architecture pipeline 100 including an artificial neural network, which may include more than one artificial neural network or sub networks, used to generate data for a 3D mesh or model of an object. The example artificial neural network shown in FIG. 1 may be considered as including into two main parts or sections. In a first part or section, the artificial neural network is predicting 2D multi-view images such as a stereo pair and in a second part or section the artificial neural network is reconstructing a 3D model of the object, in the example of FIG. 1, a face.

An embodiment uses an approach in which a simple, 2D frontal image 102 of an object such as a user's face is used to generate multiview images 104a, 104b that can be thereafter converted into a 3D model 106a. 3D model 106a, when associated with a texture 106b (data from original frontal 2D image 102 or data from synthetic 2D multiview images 104a and 104b, or a combination of the foregoing) allows for modeling features of a 3D object such as a user's face from original 2D image 102. Output of the model, e.g., facial features, some of which may be asymmetric representing differences in sides of the object, such as different features of left and right sides of a user's face, may be used for a variety of purposes. In one example, the output of the 3D model is used to match the user to a fitting category, for example for a medical device such as a respiratory mask used as a sleep or respiratory aid. In another example, output of the 3D model is used to compare the user's modelled features with stored features, e.g., in a biometric identification process, such as for example used in a login sequence or in a routine used to authorize access to a resource such as a device, application, or data.

As illustrated in FIG. 1, after capture by a camera 101, single, frontal 2D input image 102 (actual image, e.g., of a user's face) is provided to or obtained by an auto encoder 103 (encoder-decoder type architecture), which is used to generate synthetic multi-view 2D images 104a and 104b. As will be appreciated by those having skill in the art, an auto encoder 103 (encoder) is used to essentially compress frontal 2D input image 102 into latent variables that may be utilized by auto encoder 103 (decoder) to reconstruct a target such as original frontal 2D input image 102 or predetermined images, e.g., a stereo image pair (collectively indicated at 104) of synthetic multi-view 2D images 104a and 104b.

Synthetic multi-view 2D images 104a and 104b in FIG. 1 are illustrated as stereo image pair 104, with one synthetic 2D image 104a forming a left perspective picture (offset at some selected angle from frontal 2D image 102 view perspective, such as 45 degrees) and another synthetic 2D image 104b forming a right perspective picture (also offset at some selected angle from frontal 2D image 102 view perspective).

As part of generating a training set for the artificial neural network, in particular auto encoder 103, in one example computer graphics are used to synthetically generate 2D stereo image pair 104 with predetermined desirable characteristics, e.g., a wider distribution of camera distance, head pose, angle offset, and illumination as compared to frontal 2D images, e.g., image 102, captured by camera 101. However, it is noted that a training set of actual images may be used in this regard, or a mixture of actual and synthetic images, so long as desirable target outputs are at hand to train auto encoder 103.

Likewise, in one example, in addition to real or actual 2D frontal image 102 and its corresponding 3D model, an embodiment synthetically generates 2D frontal images and 2D multiview images of the same face. Therefore, for auto encoder 103 (encoder-decoder architecture) input used includes actual 2D frontal images, for example, image 102, as well as synthetically generated 2D frontal images and pipeline 100 predicts 2D multiview images 104a and 104b that can be trained using the synthetically rendered 2D multi-view training images, as will be further explained in connection with description of use of loss metrics or error correction for training auto encoder 103.

As seen in FIG. 1, the output of decoder portion of auto encoder 103 (that is, synthetic multiview images 104a and 104b) may be compared, for example, by a comparison unit 109, which may include an identity encoder to form a synthetic image of the object such as a face, or image data, for comparison to the original frontal 2D image input 102 (or data associated therewith) to compute a loss 110, useful in performing weight adjustments via back propagation to auto encoder 103. Likewise, as indicated in FIG. 1, input to comparison unit 109 may include a synthesized 2D image 108 to evaluate how pipeline 100 uses multiview images 104a and 104b.

In the example of FIG. 1, for a second part of pipeline 100 (using 2D multiview images 104a and 104b have been generated by fully or partially trained auto encoder 103), an embodiment uses a different auto encoder 105 (encoder-decoder architecture) to use generated 2D multiview images 104a and 104b to predict data 106 useful in producing a 3D model output 106. For example, an embodiment uses auto encoder 105 to provide a UV position map and 3D coordinates of the object mesh (collectively denoted at 106a). Generated 3D mesh 106a and texture 106b may be used by a model application unit 107 to produce various outputs, such as to synthetically reproduce 2D image 108, which should replicate original frontal input image 102, as well as detail the 3D characteristics of the modelled object for beneficial use in various programs, such as fitting applications, biometrics security and access control applications, etc.

Somewhat similar to the correction for loss or error in association with generating multiview 2D images 104a and 104b, in an embodiment, generated 3D data 106 may be used, e.g., by a comparison unit 111, which may again include an identity encoder, to compare against the input feature maps associated with original frontal input image 102 to compute the loss 112 from original frontal input image 102, e.g., a global loss estimation and weight adjustment (such as applied via back propagation technique for example applied to adjust weights of auto encoder 105).

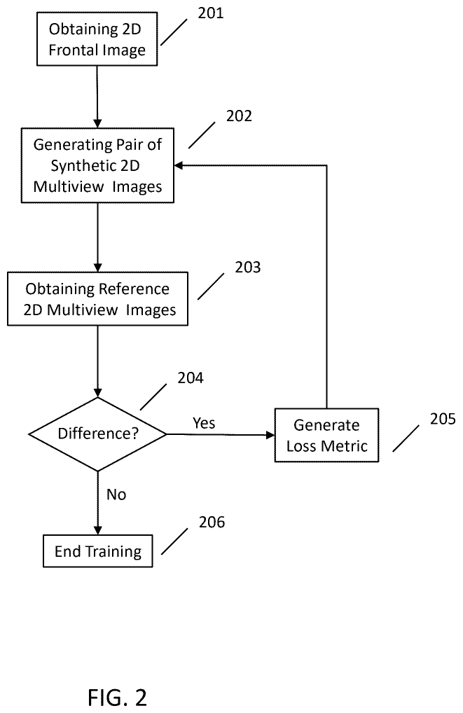

Referring to FIG. 2, with respect to the first part of pipeline 100 and training auto encoder 103 to generate suitable 2D multiview images 104a and 104b, an embodiment utilizes a method of comparing 2D multiview images 104a and 104b with reference data, e.g., synthetic or actual 2D multiview target images. By way of example, an embodiment obtains a 2D frontal image at 201 (which may be an actual or synthetic image) and generates, for example using auto encoder 103 of FIG. 1, 2D multiview images at 202. At 203 an embodiment obtains reference data, e.g., reference multiview images representing targets that are desired output of auto encoder, synthetic or actual, with which to compare at 204 the synthetically generated 2D images. For example the reference data may include data of 2D multiview images of the object at selected angular offsets for the desired application, such as facial recognition or biometric-based biomedical fitting. If the difference(s) is or are significant, as determined at 204, an embodiment determines or generates a loss metric, e.g., used for a weight update via back propagation as indicated at 205. The process of using a loss metric may be iterated until the loss minimizes or some expected performance threshold is obtained. Otherwise, the model may be considered to be sufficiently trained and the training process ended at 206. As described in connection with FIG. 1, other or additional reference data may be utilized by an embodiment to adjust a part of the network, such as using data resultant from the end of the pipeline, e.g., image 108 of FIG. 1, to adjust weights of auto encoder 103.

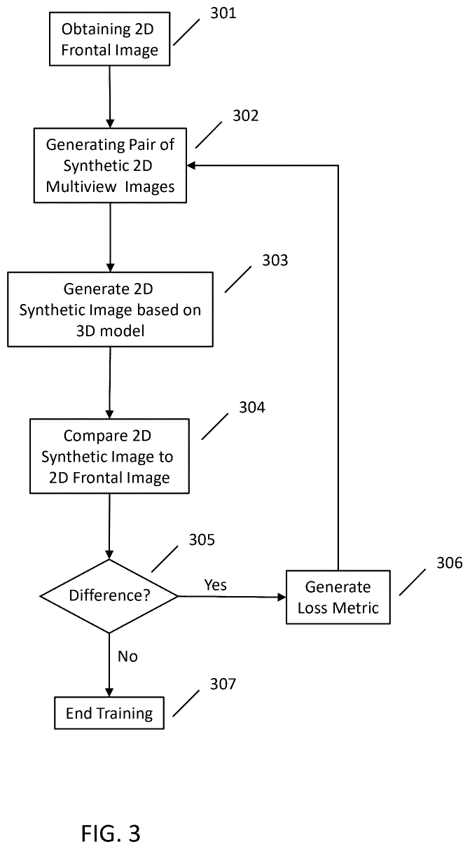

An embodiment may additionally or alternatively train the artificial neural networks, that is one or more of auto encoders 103 and 105, used in pipeline 100 by utilizing a more global loss metric, for example based on frontal input image 102. In the example of FIG. 3, an embodiment obtains a 2D frontal input image at 301 and generates a pair of multiview images at 302, similar to FIG. 2. Additionally, an embodiment uses the generated 2D multiview images to produce a 3D model, that is, the target output of auto encoder 105 of FIG. 1, and a related output, such as 2D synthetic image 108, at 303. That is, in one example, the output generated at 303 is a 2D synthetic image that can be used for training the neural network, for example compared to original 2D frontal input image 102. By way of example, an embodiment compares an output generated by the 3D model, such as comparing original and synthetically generated 2D images (or associated data) at 304 to determine, as indicated at 305, a difference. If there is a significant difference (for example above a loss or error threshold or if the loss is not yet stably minimized), then a loss metric may be generated as indicated at 306 and used for example in back propagation for training the artificial neural network via adjustment of weights of auto encoder(s), e.g., auto encoder 105 of FIG. 1. As in the example of FIG. 2, the use of a loss metric may be iterated until a minimum loss is obtained. Otherwise, the training of the artificial neural network may end, as indicated at 307.

After the artificial neural network of pipeline 100 has been sufficiently trained, an embodiment may utilize the same to produce 3D model outputs based on 2D image inputs. It will be readily apparent that the trained neural network components, such as auto encoders 103 and 105 may be exported to a device, such as provided to a mobile device, a kiosk or patient scanning device, etc., as well as implemented in a cloud or server-based computing device, such as called via an application programming interface and used to evaluate an input 2D image per the embodiments described herein.

As the pipeline 100 is trained using 2D multiview images that are purposefully selected to tune the auto encoder(s) to generate 2D synthetic multiview images, e.g., stereo image pair 104, the resultant artificial neural network component(s) such as auto encoder 103 of FIG. 1, have desirable characteristics. In one example, the desirable characteristics relate to facial asymmetry that is found in most of the population and may be utilized to advantage in fitting medical devices appropriately to a user's face or in other related applications, such as in making biometrics-based security decisions, where facial asymmetry is a useful characteristic to be preserved by a synthetically generated 3D model.

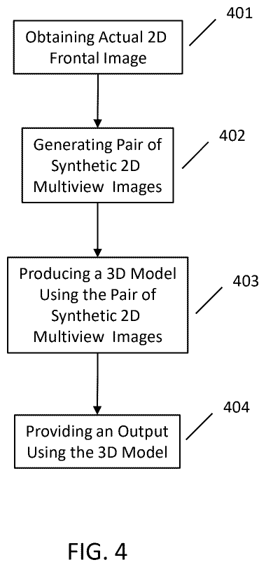

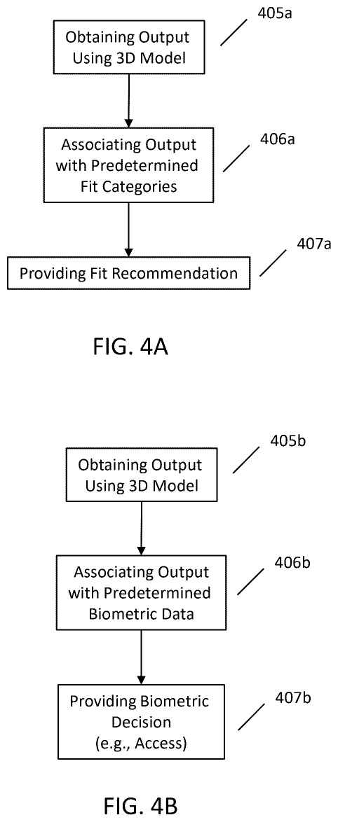

Turning to FIG. 4, a method of using a frontal 2D image and synthetically generated 2D multiview images is provided. As illustrated, a general approach may include obtaining a single 2D frontal image at 401, for example an image of a user's face captured by a smartphone, thereafter, generating a synthetic 2D multiview image pair at 402, for example a stereo pair characterizing the left and right sides of the user's face, followed by producing a 3D model at 403. This 3D model may be used to provide associated output at 404, such as feature comparison data that relate the object's known features to those represented in the 3D model or relate the object's synthesized (modelled) features to product characteristics, such as facial mask fit categories, types, etc.

Having described an example technique of selectively choosing 2D multiview images for training an artificial neural network and associated error correction techniques, it will be appreciated by those having skill in the art that the model's output may be used for several beneficial purposes not previously obtainable by conventional techniques. Highlighted in FIG. 4A and FIG. 4B are two categories of these, noting that other applications may be apparent now or become apparent in the future.

In the example of FIG. 4A it is illustrated that the output of the 3D modelling process may be provided or utilized by a fitting application, such as software used for fitting a biomedical device to the face of a user. By way of specific example, the output of the 3D model obtained at 405a may be used to associate the output with one or more predetermined fitting categories at 406a. For example, a 3D model output obtained at 405a may indicate that the user pictured in the original frontal 2D image has a particular facial asymmetry, which may be matched to a given fit category for a medical device, such as a respiratory or CPAP mask. Thereafter, the predetermined fit category may be provided as an output at 407a, e.g., as per a fitting application that delivers a fitting recommendation back to an end user such as a medical device technician or a patient.

In another example, illustrated in FIG. 4B, the 3D model output obtained at 405b may be used by a program to associate the 3D model output, or data associated therewith, with predetermined biometric data at 406b. By way of example, the output obtained at 406b, such as a model feature or numeric representation thereof may be used to compare to a known 3D feature of the user at 406b. This permits the provision, at 407b, of an output based on the association (or comparison), such as a biometric decision related to granting or denying access to a device, application, data or other resource.

From the foregoing, it will be understood that the appropriate selection of target 2D multiview images is an important consideration. This is particularly so in certain application spaces where object asymmetry cannot be glossed over or summarized, for example using a modelling process that predicts one view or partial and incomplete views of an object or its relevant surface and automatically fills in model feature details assuming the object has symmetry. According, an embodiment is specifically trained to synthesize stereo image pairs suitable for facial feature determinations, including asymmetry preservation or conservation, such as via use of stereo pairs that offer appropriate angular offsets.

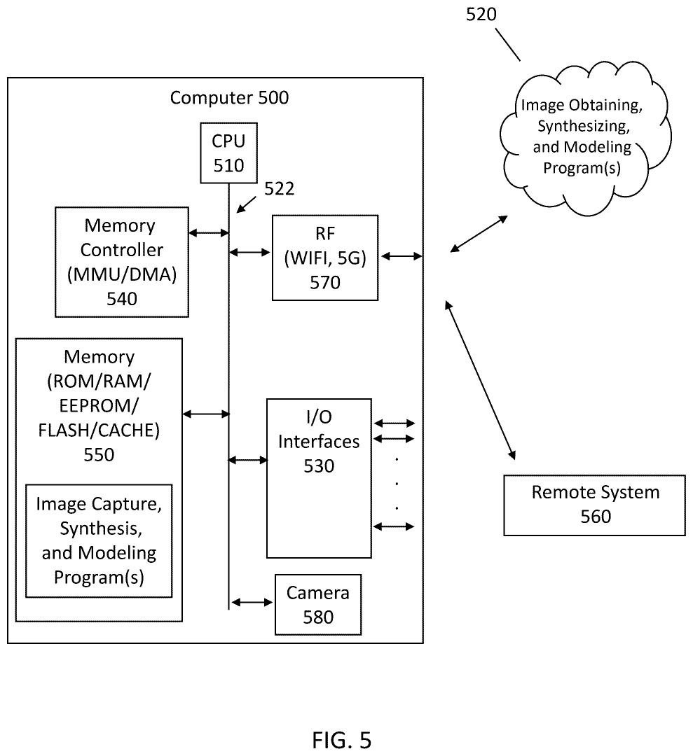

Referring to FIG. 5, it will be readily understood that certain embodiments can be implemented using any of a wide variety of devices or combinations of devices and components. In FIG. 5 an example of a computer 500 and its components is illustrated, which may be used in a device for implementing the functions or acts described herein, e.g., as a user device having a camera 580, as a modeling device, or as a remote or external device that utilizes the output of a model or comparison data derived therefrom. In addition, circuitry other than that illustrated in FIG. 5 may be utilized in one or more embodiments. The example of FIG. 5 includes certain functional blocks, as illustrated, which may be integrated onto a single semiconductor chip to meet specific application requirements.

One or more processing units are provided, which may include a central processing unit (CPU) 510, one or more graphics processing units (GPUs), and/or micro-processing units (MPUs), which include an arithmetic logic unit (ALU) that perform arithmetic and logic operations, instruction decoder that decodes instructions and provides information to a timing and control unit, as well as registers for temporary data storage. CPU 510 may comprise a single integrated circuit comprising several units, the design and arrangement of which vary according to the architecture chosen.

Computer 500 also includes a memory controller 540, e.g., comprising a direct memory access (DMA) controller to transfer data between memory 550 and hardware peripherals such as camera 580. Memory controller 540 includes a memory management unit (MMU) that functions to handle cache control, memory protection, and virtual memory. Computer 500 may include controllers for communication using various communication protocols (e.g., I²C, USB, etc.).

Memory 550 may include a variety of memory types, volatile and nonvolatile, e.g., read only memory (ROM), random access memory (RAM), electrically erasable programmable read only memory (EEPROM), Flash memory, and cache memory. Memory 550 may include embedded programs, code and downloaded software, e.g., artificial neural network program(s) trained using select 2D synthetic or actual multiview images useful in producing predetermined, differential 2D multiview image outputs for use in 3D models as described herein. By way of example, and not limitation, memory 550 may also include an operating system, application programs, other program modules, code and program data, which may be downloaded, updated, or modified via remote devices.

A system bus 522 permits communication between various components of the computer 500. I/O interfaces 530 and radio frequency (RF) devices 570, e.g., WIFI and telecommunication radios, may be included to permit computer 500 to send and receive data to and from remote devices using wireless mechanisms, noting that data exchange interfaces for wired data exchange may be utilized. Computer 500 may operate in a networked or distributed environment using logical connections to one or more other remote computers or databases. The logical connections may include a network, such local area network (LAN) or a wide area network (WAN), but may also include other networks/buses. For example, computer 500 may communicate data with and between a device 520 running one or more artificial neural networks, training programs for training the same, and other devices 560, e.g., a remote system that uses data such as a fit parameter, biometric match decision, etc., as described herein. It will be appreciated by those having skill in the art that artificial neural networks such as those described herein, once trained, may be provided and used on a local device, e.g., computer 500, which may take the form of an end user device such as a smartphone, tablet, desktop computer, etc.

Computer 500 may therefore execute program instructions or code configured to generate, store and analyze 3D model output data based on 2D image input and perform other functionality of the embodiments, as described herein. A user can interface with (for example, enter commands and information) the computer 500 through input devices, which may be connected to I/O interfaces 530. A display or other type of device may be connected to the computer 500 via an interface selected from I/O interfaces 530.

It should be noted that the various functions described herein may be implemented using instructions or code stored on a memory, e.g., memory 550, that are transmitted to and executed by a processor, e.g., CPU 510. Computer 500 includes one or more storage devices that persistently store programs and other data. A storage device, as used herein, is a non-transitory computer readable storage medium. Some examples of a non-transitory storage device or computer readable storage medium include, but are not limited to, storage integral to computer 500, such as memory 550, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations according to various embodiments may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections or through a hard wire connection, such as over a USB connection.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method, comprising:
obtaining, using a set of processors, an actual, frontal two-dimensional (2D) image of an object;
generating, using the set of processors, a pair of synthetic 2D multiview images of the object based on the actual, frontal 2D image of the object;
producing, using the set of processors, a three-dimensional (3D) model of the object based on at least the pair of synthetic 2D multiview images;
wherein the 3D model conserves an asymmetry of the object; and
providing, using the set of processors, an output using the 3D model of the object that conserves the asymmetry.

2. The method of claim 1, wherein the pair of synthetic 2D multiview images comprise a stereo pair of 2D synthetic images.

3. The method of claim 2, wherein the stereo pair of 2D synthetic images comprise a stereo pair having a predetermined angular offset.

4. The method of claim 3, wherein the predetermined angular offset is selectable.

5. The method of claim 5, comprising selecting a training stereo pair of actual images of an object having the predetermined angular offset.

6. The method of claim 1, wherein the generating comprises using an artificial neural network to generate the pair of synthetic 2D multiview images of the object;
the artificial neural network having been trained using corresponding actual or synthetic 2D multiview images of representative objects.

7. The method of claim 6, wherein the artificial neural network comprises a set of auto encoders;
the method comprising comparing an output of one or more decoders to an original input frontal 2D image to generate an error metric for training the artificial neural network.

8. The method of claim 1, wherein the actual, frontal 2D image is an image of a face.

9. The method of claim 8, wherein the output comprises one of an access decision and a fit recommendation.

10. The method of claim 9, wherein the fit recommendation is a fit recommendation for a sleep therapy mask.

11. A device, comprising:
a set of one or more processors; and
a set of one or more memory devices operatively coupled to the set of one or more processors and storing code executable by the set of one or more processors to:
obtain an actual, frontal two-dimensional (2D) image of an object;
generate a pair of synthetic 2D multiview images of the object based on the actual, frontal 2D image of the object;
produce a three-dimensional (3D) model of the object based on at least the pair of synthetic 2D multiview images;
wherein the 3D model conserves an asymmetry of the object; and
provide an output using the 3D model of the object that conserves the asymmetry.

12. The device of claim 11, wherein the pair of synthetic 2D multiview images comprise a stereo pair of 2D synthetic images.

13. The device of claim 12, wherein the stereo pair of 2D synthetic images comprise a stereo pair having a selectable, predetermined angular offset.

14. The device of claim 13, comprising a camera configured to obtain the actual, frontal 2D image.

15. A computer program product, comprising:
a non-transitory storage medium comprising computer executable code, the computer executable code comprising:
code that obtains an actual, frontal two-dimensional (2D) image of an object;
code that generates a pair of synthetic 2D multiview images of the object based on the actual, frontal 2D image of the object;
code that produces a three-dimensional (3D) model of the object based on at least the pair of synthetic 2D multiview images;
wherein the 3D model conserves an asymmetry of the object; and
code that provides an output using the 3D model of the object that conserves the asymmetry.
